(19) United States
(12) Patent Application Publication
(10) Pub. No.: US 2015/0157490 A1
(43) Pub. Date: Jun. 11, 2015

(54) DRAIN POUCH CADDY

(71) Applicants: Sandra Sue Wellborn, Arlington, TX (US); Jamie Elaine Hildreth, Arlington, TX (US)

(72) Inventors: Sandra Sue Wellborn, Arlington, TX (US); Jamie Elaine Hildreth, Arlington, TX (US)

(21) Appl. No.: 14/100,659

(22) Filed: Dec. 9, 2013

(51) Int. Cl. A61F 5/44

(52) U.S. Cl. CPC A61F 5/4408

(57) ABSTRACT

The Drain Pouch Caddy provides a way to contain, position, hold, and/or support a pouch drainage and tube system following surgery to the upper portion of a body in which a stoma is created in order for drain tubes to be inserted under the skin. In particular, the Drain Pouch Caddy is a narrow, fabric neck scarf in which both ends are secured into a loop with the use of a button and button hole to contain, position, hold, and support up to six drain pouches and tubes. The Drain Pouch Caddy is appropriate for use when resting, walking, engaging in activities and showering.

### FIELD OF THE INVENTION

The Drain Pouch Caddy is a narrow neck scarf constructed of soft, washable fabric with a fastening system on each end for containing, positioning, holding, and/or supporting up to six drainage pouch and tube systems attached on a body via surgical stomas.

### BACKGROUND OF THE INVENTION

Single or bilateral mastectomies and lumpectomies as well as other surgical procedures often require the use of one or more fluid gathering pouches attached to the body with drainage tubes 2-feet or longer. The drain pouches generally have a loop attached to enable the patient to secure the drains to clothing or other device by looping or pinning with a safety or diaper pin. Safety, mobility, dignity and bathing are issues that concern patients and medical staff. The drain pouches should be contained discretely in such a way as to allow the drain pouches be accessed, measured and drained easily as well as for the fluids to drain freely. Tubes should be secured in such a way as to keep the tubes from catching on protuberances such as door knobs that may pull the drains out of the stoma, creating sterility issues and possibly necessitating further surgery in order to reattach the tubes. One of the biggest issues for a patient has been how secure the drains and tubes in order to take a shower while the drains are attached.

A Drain Pouch Caddy is an aid that provides a means for drain pouches and tubes to be contained and secured while at rest, mobile or bathing. The device, being narrow and a cloth material, dries quickly and easily. The fastening system closing makes it easy for almost anyone to secure and unsecure the ends of the Pouch Caddy.

The weight of a Drain Pouch Caddy is less than one ounce in order to create the least discomfort, irritation and/or soreness to the patient.

### SUMMARY

A Drain Pouch Caddy is designed for patients with one or more surgically created stomas to easily and comfortable contain and secure the attached drains and tubes that have been inserted through the skin so fluids may be drained from the body. Use of the device allows the tubes and pouch be looped through the ends of the device and secured. Resting, walking and bathing are easily accomplished with the Caddy, yet allow the patient dignity and safety as the Caddy can be worn under clothing thus hiding the pouches and drains.

### BRIEF DESCRIPTION OF THE DRAWINGS

The following summary and detailed description is better understood when read in conjunction with the accompanying drawings which are included by way of example and not by way of limitation.

FIG. 1 shows a drawing of the front of the Drain Pouch Caddy showing the button on each end and the button hole approximately 5 inches from the end.

FIG. 2 shows a drawing of the back of the Drain Pouch Caddy.

FIG. 3 shows a drawing of the Drain Pouch Caddy looped around a patient's neck with the ends closed using the fastening system.

FIG. 4 shows a drawing of the Drain Pouch Caddy looped around a patient's neck with two drains secured by the Drain Pouch Caddy—one drain and attached tubes on each side of the torso. Closure is accomplished with the use of a button and button hole fastening system.

FIG. 5 shows a drawing of the Drain Pouch Caddy looped around a patient's neck securing the “empty” end of the Drain Pouch Caddy to the “full” end by the fastening system.

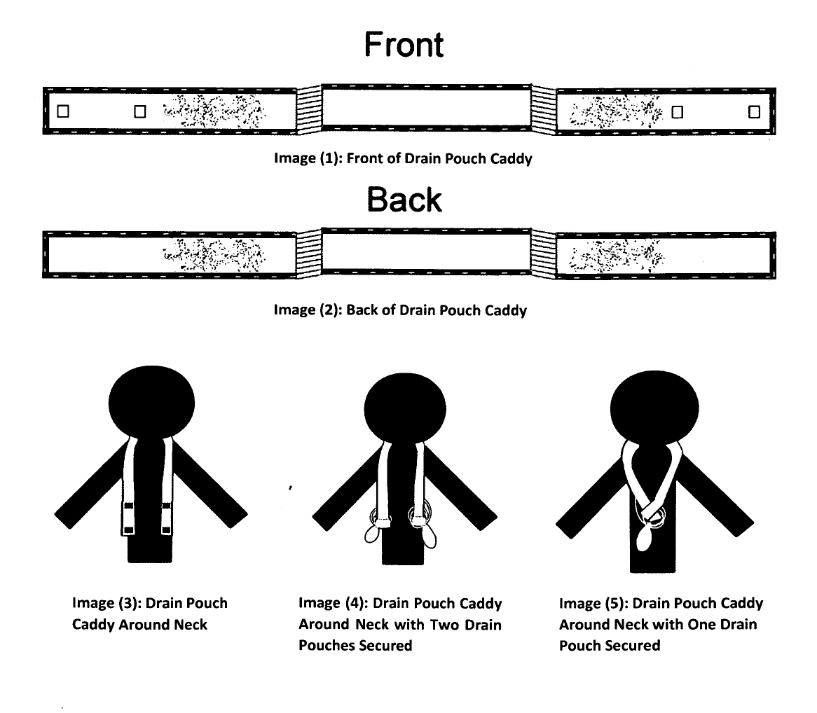

In FIGS. 1 and 2, the drawings of broken lines form no part of the claimed design as the Drain Pouch Caddy is constructed in several lengths and indicate that the precise length of the scarf is not claimed.

### DETAILED DESCRIPTION OF EMBODIMENTS

The Drain Pouch Caddy provides a method to contain, position, hold, and/or support up to six pouches drainage pouch and tubing assembly while resting, walking, engaging in activities and bathing for post-operative patients with one or more stomas.

In some embodiments, the component length is sized appropriately for the height of the patient (e.g. 46 inch, 44, inch, 42 inch, 40 inch, 38 inch, 36 inch, 38 inch, 36 inch, 34 inch, 32 inch, etc.). In all embodiments, the device secures standard and/or mini-pouches. In some embodiments, the tube component may be secured with the Drain Pouch Caddy.

In some embodiments, the fastening system is comprised of a button and button holes closure system In some embodiments, the component length is sized appropriately for the height of the patient (e.g. 46 inch, 44, inch, 42 inch, 40 inch, 38 inch, 36 inch, 38 inch, 36 inch, 34 inch, 32 inch, etc.). In all embodiments, the device secures standard and/or mini-pouches. In some embodiments, the tube component may be secured with the Drain Pouch Caddy.

In some embodiments, the Drain Pouch Caddy relieves emotional stress of a subject by freeing them from worry about their drain pouch and tube system. In some embodiments, the present invention can be worn by the patient at any time after surgery, allowing the patient mobility without having to carry the pouches by hand or by another method. The Drain Pouch Caddy can be worn under the patient's normal outer garments. In some embodiments, the present invention can provide support of draining and tubing system(s) for a subject of any size, age, height or weight.

### PATENT CITATIONS

| Cited Patent | Filing date | Applicant | Title |
|---|---|---|---|
| 4,037,340 | 10-Feb-76 | Alan Sidney Lewis | Scarf |
| | | Owensmith | |
| 4,109,321 | 3-Jan-77 | Rosella Shapiro | Scarf constructions having a substantially |
| | | | concealable pocket therein, and methods of |
| | | | producing same |
| 4,220,302 | 21-Jul-78 | Hampton, Diane M | Nursing bottle holder |
| 4,321,709 | 13-Jan-81 | Ray Steinberg | Decorative scarf |
| 4,337,938 | 6-Mar-81 | Rodriguez; Basilides | Jogger's aid |
| 4,341,331 | 27-Jul-81 | Mcdougall; Patrick P. | Article holder |
| 4,404,688 | 3-Feb-82 | Phyllis Knight | Combination scarf |
| 4,498,613 | 11-Oct-83 | Donahue; Barbara S. | Support device for nursing bottles and amusement |
| | | | items |
| 4,498,615 | 26-Aug-83 | Edward J. Erpelding | Golf tee holder |
| 4,537,341 | 2-Aug-84 | Kelly; Oscar | Nursing bottle holder |
| 4,631,754 | 14-Dec-84 | Barbara D. Ryan | Nursing scarf |
| 4,858,801 | 14-Mar-88 | Sameniego; Manuel | Guitar strap |
| 4,870,707 | 27-May-88 | Alia S. Hayes | Multiple-purpose scarf |
| 4,911,347 | 28-Sep-88 | Wilhite; Daniel W. | Carrier and locking seal for articulated drawing |
| | | | tubes and other cylindrical objects with slip on end |
| | | | caps |
| 4,942,627 | 8-May-89 | Judy Copprue | Scarf |
| 4,978,044 | 24-May-89 | Ruth Silver And | Slidably engaged luggage strap |
| | | Company | |
| 5,016,288 | 5-Sep-90 | Peter Pan | Scarf |
| 5,025,508 | 18-Dec-89 | Patty S. Duncan | Scarf construction |
| 5,033,612 | 21-Jun-90 | Elaine Bivins | Eyeglass and scarf holder |
| 5,205,832 | 7-May-92 | Tuman; David H. | Endo-tracheal tube support device |
| 5,291,616 | 29-Jan-93 | Susan B. Lamons and | Waistband accessory device and wrapping method |
| | | Lisa A. Rhoades | using a scarf and a scarf shaper form |
| 5,332,137 | 5-May-93 | Violette; Michael | Musical instrument harness |
| 5,643,233 | 1-Nov-95 | Nancy F. Turner | Post-surgical dranage container carrier |
| 5,664,581 | 17-Sep-96 | Ashley; John P. | Intravenous tubing secure strap |
| 5,720,049 | 28-Sep-95 | Geoffrey Clutton | Scarf |
| 5,722,573 | 6-Aug-96 | Carnel; Christopher Paul | Portable system for delivering a drinking beverage |
| 6,006,970 | 26-Aug-98 | Piatt; John Allen | Computer harness to support a computer about the |
| | | | body of a user operator |
| 6,032,292 | 29-Jan-99 | Chester Jame and Betty | Convertible bandanna or scarf |
| | | Hane Wood | |
| 6,152,915 | 23-Mar-99 | Paul L. and Viola M. | Drain tube belt and shower pack kit |
| | | Watson | |
| 6,226,799 | 23-May-00 | Barry Lane | Scarf |
| 6,270,485 | 8-Apr-99 | Barbara Norton Ekey | Post Surgical drain receptacle support system |
| 6,296,164 | 13-Jul-99 | Ronald D. Russo | Medical Device Holder |
| 6,371,346 | 2-Jun-99 | Sanjiv Ranjan Sharma | Interchangeable equipment carrier sling/waist belt |
| 6,511,467 | 12-Sep-00 | Denise Reuss | Drainage reservoir garment |
| 6,524,288 | 24-Aug-01 | Michelle Hadley-Fruit | Drainage reservoir support assembly |
| 6,610,032 | 21-Aug-00 | Dale Medical, Inc. | Surgical drainage device |
| 6,610,032 | 21-Aug-00 | Mary Ann Robinson | Surgical drainage device |
| | | Prody | |
| 6,979,303 | 2-Sep-03 | Bernadette Jestrabek- | Arm sling apparatus allowing movement or total |
| | | Hart | immobilization |
| 7,048,160 | 25-Feb-03 | Anderson Antonio D | Infant feeding and entertainment support |
| 7,231,670 | 27-Apr-06 | Lily Y. Chang | Versatile scarf |
| 7,892,621 | 21-Oct-08 | Mikel Draghici, MD John | Container scarf |
| | | Ingrassia, MD | |
| 7,927,311 | 5-Dec-07 | Deborah Bachelder | Post-surgica drainage bulb support sling |
| 8,226,621 | 31-Jul-08 | Timmons Vicki C | Urine collection bag support belt |
| 8,292,860 | 8-Dec-08 | Persichetti Gwen C | Medical drainage pouch |
| 8,468,612 | 15-Feb-11 | Marie A. Galloway | Scarf system with sleeve pockets |
| 20,040,144,820 | 28-Jan-03 | Jeffrey Herold | Modular dual shoulder strap for golf club bags |
| 20,080,296,325 | 29-May-08 | Gina Tepper | Bottle/can tethers |
| 20,090,062,705 | 2-Sep-08 | Victor Toso | Adjustable back support device |
| 20,100,249,734 | 25-Mar-10 | Ostomy Living, Llc | Ostomy pouch containing and supporting device and |
| | | | system |

CLASSIFICATIONS

| | |
|---|---|
| U.S. Classification | 602/4, 604/345, 604/179, 224/257, 224/578, 224/600, 224/602, 224/605, |
| | 224/901.2 |
| International | A45C13/30, A45C15/00, A45F3/00, A45F5/00, A61F2/00, A61F5/44, |
| Classification | A61M5/32, A61F27/00 |
| Cooperative | A45F5/00, A45F2003/142, A45F2005/006, A61F5/4408 |
| Classification | |
| European Classification | A61F27/00, A45F5/00 |

1) The Drain Pouch Caddy—a narrow neck scarf worn around the neck—contains, positions, holds, and supports a total of one-to-six pouch drainage and tube system(s) following surgery to the upper portion of a body in which a stoma is created in order for drain tubes to be inserted through the skin.
The Drain Pouch Caddy has the following attributes:
The Drain Pouch Caddy is placed around the neck of the post-operative patient and the pouches are secured to the ends of the Caddy. The Caddy may be worn while at rest, mobile, engaging in activities or in the shower. The Drain Pouch Caddy is constructed of a soft, lightweight, hand-washable material that dries quickly by air. Ideally, having two Caddies lets one be worn while the other is drying after washing or showering
Fastening closures are at both ends of the Drain Pouch Caddy to easily and quickly secure the drainage pouches (See Drawings 1 and 2).
The drain pouches have a strong fastening system of button and button hole. The Drain Pouch Caddy is worn around the neck like a scarf (See Drawing 3.) One tab of the support is slipped through up to three (3) drain pouch loops on one side. The Caddy tab is brought up and secured by the fastening system. The remaining drain pouches, up to three (3) on the other side, are secured to the opposite end of the Caddy in the same manner as the first (See Drawing 4). Should there be only one drain pouch or the drain pouches are only on one side of the body, one tab of the support is slipped through the drain pouch loop(s) on that side. The Caddy tab is brought up and secured by the fastening system. The empty support tab is looped through the first tab and the two Caddy tabs are secured together by the fastening system (See drawing 5).
Pouches dangling from long tubes emanating from the skin and attached with stitches are difficult for most post-operative patients to deal with. The tubes can get caught on doorknobs or other protuberances and the drain pouches can fall leading to painful and dangerous pulling out of the tubes, especially in the shower. The Drain Pouch Caddy is designed to aid the post-operative patient to live more comfortably with the drain pouches.
The Drain Pouch Caddy is designed for post-operative patients with most levels of dexterity to easily attach and release the support tabs.
Use of the Drain Pouch Caddy allows the tubes and pouch be looped through the ends of the device and secured with the fastening system. (See Drawing 4)
A standard length Drain Pouch Caddy is approximately 47″ long.
